(19) United States
(12) Patent Application Publication
(10) Pub. No.: US 2008/0065073 A1
(43) Pub. Date: Mar. 13, 2008

(54) OFFSET DYNAMIC MOTION SPINAL STABILIZATION SYSTEM

(76) Inventors: Michael Perriello, Hopedale, MA (US); Dennis Colleran, North Attleboro, MA (US); Arnold Oyola, Northborough, MA (US); Sally Carter, Willingford, CT (US); Joshua Morin, Sturbridge, MA (US)

Correspondence Address: CARR LLP (IST), 670 FOUNDERS SQUARE, 900 JACKSON STREET, DALLAS, TX, 75202, US

(21) Appl. No.: 11/852,821

(22) Filed: Sep. 10, 2007

Related U.S. Application Data
(60) Provisional application No. 60/825,078, filed on Sep. 8, 2006.
(60) Provisional application No. 60/826,807, filed on Sep. 25, 2006.
(60) Provisional application No. 60/826,817, filed on Sep. 25, 2006.

(51) Int. Cl. A61B 17/58

(52) U.S. Cl. 606/61; 606/100

(57) ABSTRACT

Provided is an offset dynamic motion system. In one example, the system includes an offset member having a rod connecting a shaped portion to a threaded portion, where a longitudinal axis of the threaded portion is angled relative to a longitudinal axis of the rod and the shaped portion is configured to couple to a polyaxial head. A first dynamic member is configured to rotationally couple to another polyaxial head. A second dynamic member is configured to rotationally couple to the threaded end of the offset member and to slideably receive part of the first dynamic member.

### CLAIM OF PRIORITY AND CROSS-REFERENCE TO RELATED APPLICATIONS

This application claims priority from U.S. Provisional Patent Application Ser. No. 60/825,078, filed on Sep. 8, 2006, U.S. Provisional Patent Application Ser. No. 60/826,807, filed on Sep. 25, 2006, and U.S. Provisional Patent Application Ser. No. 60/826,817, filed on Sep. 25, 2006, all of which are incorporated by reference herein in their entirety.

This application is related to U.S. patent application Ser. No. 11/693,394, filed on Mar. 29, 2007, which is incorporated by reference herein in its entirety.

### FIELD OF THE INVENTION

This disclosure relates to skeletal stabilization and, more particularly, to systems and method for stabilization of human spines and, even more particularly, to dynamic stabilization techniques.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis into flexion (bending forward or anterior) and extension (bending backward or posterior), vertebrae of the spine must rotate about the horizontal axis to various degrees of rotation. The sum of all such movement about the horizontal axis of produces the overall flexion or extension of the spine. For example, the vertebrae that make up the lumbar region of the human spine move through roughly an arc of 3° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 3° in flexion and about 5° in extension if in the lumbar region) centered about a center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together while the anterior edges move farther apart thereby compressing the posterior of the spine. During flexion and extension the vertebrae move in horizontal relationship to each other providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite that portion compressed is expanded, increasing the spacing between the edges of vertebrae comprising that portion of the spine. For example, the vertebrae that make up the lumbar region of the human spine rotate about an axis of roll, moving through an arc of around 10° relative to its neighbor vertebrae throughout right and left lateral bending.

Rotational movement about a vertical axis relative is also natural in the healthy spine. For example, rotational movement can be described as the clockwise or counter-clockwise twisting rotation of the vertebrae during a golf swing.

In a healthy spine the inter-vertebral spacing between neighboring vertebrae is maintained by a compressible and somewhat elastic disc. The disc serves to allow the spine to move about the various axes of rotation and through the various arcs and movements required for normal mobility. The elasticity of the disc maintains spacing between the vertebrae during flexion and lateral bending of the spine thereby allowing room or clearance for compression of neighboring vertebrae. In addition, the disc allows relative rotation about the vertical axis of neighboring vertebrae allowing twisting of the shoulders relative to the hips and pelvis. A healthy disc further maintains clearance between neighboring vertebrae thereby enabling nerves from the spinal chord to extend out of the spine between neighboring vertebrae without being squeezed or impinged by the vertebrae.

In situations where a disc is not functioning properly, the inter-vertebral disc tends to compress thereby reducing inter-vertebral spacing and exerting pressure on nerves extending from the spinal cord. Various other types of nerve problems may be experienced in the spine, such as exiting nerve root compression in the neural foramen, passing nerve root compression, and ennervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each in order to maintain space for the nerves to exit without being impinged upon by movements of the spine.

In one such procedure, screws are embedded in adjacent vertebrae pedicles and rigid rods or plates are then secured between the screws. In such a situation, the pedicle screws press against the rigid spacer which serves to distract the degenerated disc space thereby maintaining adequate separation between the neighboring vertebrae to prevent the vertebrae from compressing the nerves. Although the foregoing procedure prevents nerve pressure due to extension of the spine, when the patient then tries to bend forward (putting the spine in flexion), the posterior portions of at least two vertebrae are effectively held together. Furthermore, the lateral bending or rotational movement between the affected vertebrae is significantly reduced, due to the rigid connection of the spacers. Overall movement of the spine is reduced as more vertebras are distracted by such rigid spacers. This type of spacer not only limits the patient's movements, but also places additional stress on other portions of the spine, such as adjacent vertebrae without spacers, often leading to further complications at a later date.

In other procedures, dynamic fixation devices are used. However, conventional dynamic fixation devices do not facilitate lateral bending and rotational movement with respect to the fixated discs. This can cause further pressure on the neighboring discs during these types of movements, which over time may cause additional problems in the neighboring discs.

Accordingly, dynamic systems which approximate and enable a fuller range of motion while providing stabilization of a spine are needed.

### SUMMARY

In one embodiment, a dynamic stabilization device having an integrated offset comprises a first member and a second member. The first member has first and second portions aligned along a longitudinal axis, wherein the first portion is configured to rotationally couple to a first polyaxial head and includes a first intersecting axis that extends through the first portion at an angle to the longitudinal axis to intersect a center point. The second member has a third portion aligned along the longitudinal axis and slideably engaging the second portion, and a fourth portion offset from the longitudinal axis and configured to rotationally couple to a second polyaxial head, the fourth portion including a second intersecting axis that extends through the fourth portion at an angle to the longitudinal axis to intersect the center point, wherein the longitudinal axis is curved to maintain the intersection of the first and second intersecting axes with the center point as the center point moves along a curved three dimensional surface during movement of the first member relative to the second member.

In another embodiment, a dynamic stabilization system having an offset member for a single dynamic device comprises an offset member, a first dynamic member, and a second dynamic member. The offset member has a rod connecting a shaped first portion to a threaded second portion, wherein a first longitudinal axis of the threaded second portion is angled relative to a second longitudinal axis of the rod, and wherein the shaped first portion is configured to couple to a first polyaxial head. The first dynamic member has first and second portions oriented along a third longitudinal axis, wherein the first portion is configured to rotationally couple to a second polyaxial head and includes a first intersecting axis that extends through the first portion at an angle to the third longitudinal axis to intersect a center point. The second dynamic member has third and fourth portions oriented along the third longitudinal axis, wherein the third portion is configured to rotationally couple to the threaded second end of the offset member and includes a second intersecting axis that extends through the third portion at an angle to the third longitudinal axis and along the first longitudinal axis of the threaded second end to intersect the center point, wherein the fourth portion is configured to slideably receive the second portion, and wherein the first and second dynamic members are configured to maintain the intersection of the first and second intersecting axes with the center point as the center point moves along a curved three dimensional surface during movement of the first dynamic member relative to the second dynamic member.

In yet another embodiment, a dynamic stabilization system having an offset member for multiple dynamic devices comprises an offset member, a first dynamic device, and a second dynamic device. The offset member has a rod with a first end coupled to a first polyaxial head, a second end coupled to a second polyaxial head, and first and second threaded extensions extending substantially perpendicularly to a longitudinal axis of the rod between the first and second ends. The first dynamic device has a first member rotatably coupled to the first threaded extension and slideably engaged to a second member of the first dynamic device that is coupled to a third polyaxial head, wherein movement of the first member relative to the second member and the offset member defines movement of a first center point along a first curved three dimensional surface. The second dynamic device has a third member rotatably coupled to the second threaded extension and slideably engaged to a fourth member of the second dynamic device that is coupled to a fourth polyaxial head, wherein movement of the third member relative to the fourth member and the offset member defines movement of a second center point along a second curved three dimensional surface.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a perspective view of an embodiment of a dynamic stabilization system;

FIG. 2 is a cross-sectional view of one embodiment of the dynamic stabilization system of FIG. 1;

FIG. 3A is an exploded view of one embodiment of a locking assembly that may be used with the dynamic stabilization system of FIG. 1;

FIG. 3B is a cross-sectional view of one embodiment of the locking assembly of FIG. 3A in an assembled state;

FIGS. 4 and 5 are a cross-sectional view of one embodiment of the dynamic stabilization system of FIG. 1; and

FIG. 6 is a perspective view of one embodiment of the dynamic stabilization system of FIG. 1.

FIG. 7 is a perspective back view of another embodiment of a dynamic stabilization system;

FIG. 8 is a back view of one embodiment of a dynamic stabilization device that may be used in the dynamic stabilization system of FIG. 7;

FIG. 9 is a side view of the dynamic stabilization device of FIG. 8;

FIG. 10 is a cross-sectional view of one embodiment of the dynamic stabilization device of FIGS. 8 and 9 taken along lines A-A of FIG. 8;

FIGS. 11A and 11B are top and side views, respectively, of one embodiment of an upper member of the dynamic stabilization device of FIG. 8;

FIG. 12A is an embodiment of an anchor portion of the upper member of FIGS. 11A and 11B taken along lines A-A of FIG. 11A.

FIG. 12B is an embodiment of a bearing element that may be used in the anchor portion of FIG. 12A.

FIG. 12C is an embodiment of a collet that may be used in the anchor portion of FIG. 12A.

FIG. 12D is an embodiment of a bushing ring that may be used in the anchor portion of FIG. 12A.

FIG. 13 is a top view of an embodiment of a lower member of the dynamic stabilization device of FIG. 8;

FIGS. 14A and 14B are perspective and top views, respectively, of an embodiment of a cover attachment band that may be used with the dynamic stabilization device of FIG. 8;

FIG. 15 is a perspective view of one embodiment of a tension band that may be used with the dynamic stabilization device of FIG. 8.

FIG. 16 is a perspective view of one embodiment of an extension bumper that may be used with the dynamic stabilization device of FIG. 8.

FIG. 17 is a perspective view of one embodiment of a bearing post that may be used with the dynamic stabilization device of FIG. 8.

FIG. 18 is a perspective view of one embodiment of a stop pin that may be used with the dynamic stabilization device of FIG. 8.

FIG. 19 is a back view of one embodiment of a dynamic stabilization device of FIG. 8 with surgical components.

FIG. 20 is cross-sectional side view of the dynamic stabilization device of FIG. 19 taken along lines A-A.

FIG. 21 is another perspective view of the dynamic stabilization system of FIG. 7.

FIG. 22 is a side view of the dynamic stabilization system of FIG. 7.

FIGS. 23A-23F are cross-sectional views illustrating shaft/sliding portion interaction between upper and lower members in various embodiments of the dynamic stabilization system of FIG. 7.

FIG. 24 is a perspective view of one embodiment of a dynamic stabilization device with a rod offset.

FIG. 25 is a perspective view of another embodiment of the dynamic stabilization device of FIG. 24 with a rod offset.

FIGS. 26 and 27 are perspective and side views, respectively, of another embodiment of a dynamic stabilization device with a rod offset.

FIG. 28 is a perspective view illustrating the dynamic stabilization devices of FIGS. 24 and 26.

FIG. 29 is a perspective view of another embodiment of a dynamic stabilization system with a rod offset.

FIG. 30 is a perspective view of still another embodiment of a dynamic stabilization system with a rod offset.

### DETAILED DESCRIPTION

It is to be understood that the following disclosure provides many different embodiments, or examples, for implementing different features of the disclosure. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

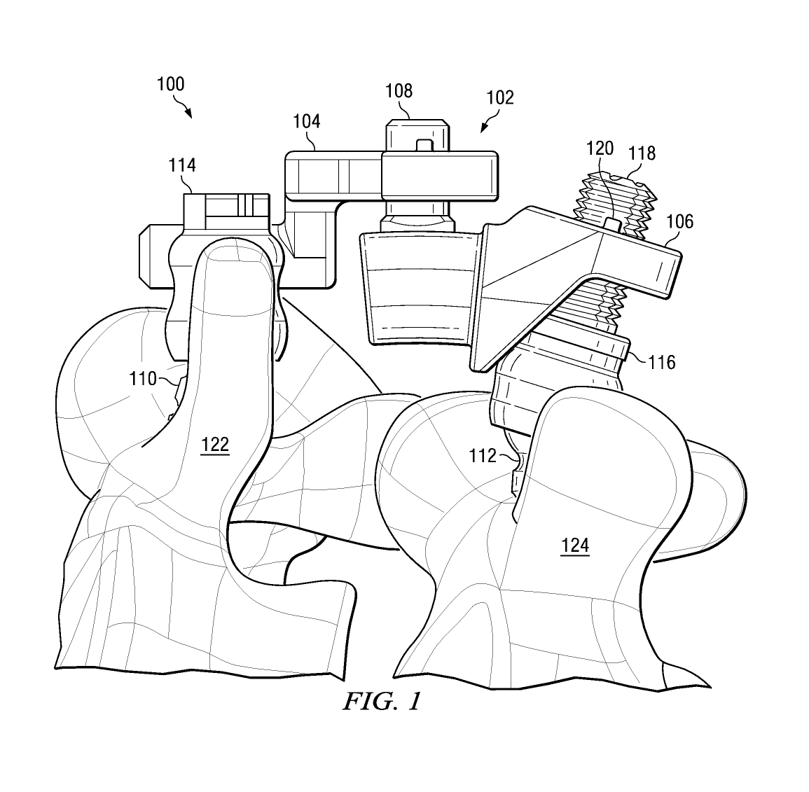

Referring to FIG. 1, in one embodiment, a spine stabilization system 100 is illustrated. The spine stabilization system 100 may be fitted to varying anatomies while providing a consistent range of motion, consistent dampening forces at the extremes of motion, alignment with a desired center of rotation (e.g., 60-70% A-P), and co-alignment of left and right systems. For example, the spine stabilization system 100 may provide height adjustment, spherical functionality, and/or sliding adjustment for variations in a patient's anatomy.

The dynamic stabilization device 102 may include two anchor members 104 and 106 coupled by a sliding member 108. The sliding member 108 may enable the two anchor members 104 and 106 to move with respect to one another, as will be described later in greater detail.

Each anchor member 104 and 106 may be secured to a portion of a vertebral body 122 and 124, respectively, such as a pedicle, via a fastening element such as a bone anchor (e.g., a pedicle screw) 110 and 112, respectively. In the present example, each bone anchor 110 and 112 may include or be coupled to a polyaxial head 114 and 116, respectively. The anchor members 104 and 106 may then be coupled to their respective polyaxial head 114 and 116 to link each anchor member with a bone anchor. For example, the polyaxial head 114 may include a slot or other opening for receiving a portion of the anchor member 104. The polyaxial head 116 may be configured to receive a bearing post 118 (e.g., a locking screw), and the anchor member 106 may couple to the polyaxial head via the bearing post and a threaded bearing element 120. It is understood that while the present example illustrates different configurations for coupling the anchor members 104 and 106 to their respective polyaxial heads 114 and 116, a single configuration may be used in some embodiments.

Although not shown, the polyaxial heads 114 and 116 and/or the anchor members 104 and 106 may be aligned with a center of rotation as described with respect to the dynamic stabilization device 100 of FIG. 1. Accordingly, two and three dimensional movement of the anchor members 104 and 106 may be constrained to ensure that axes of the polyaxial heads 114 and 116 and/or the anchor members 104 and 106 remain aligned with the center of rotation.

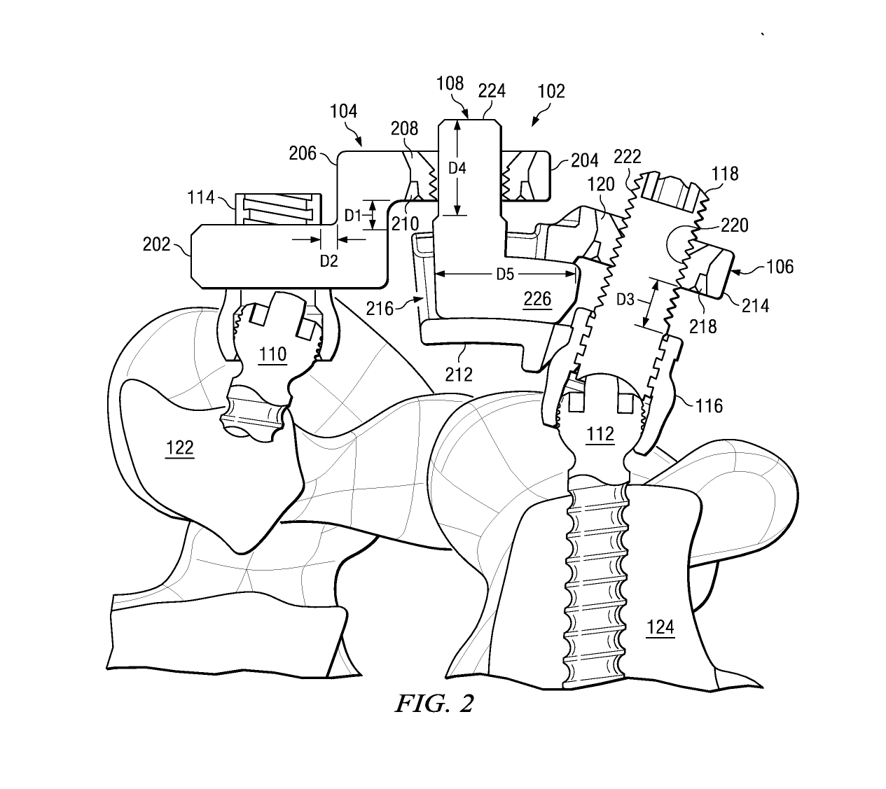

Referring to FIG. 2, a cross-sectional view of one embodiment of the dynamic stabilization device 102 of FIG. 1 is illustrated. As stated with respect to FIG. 1, the dynamic stabilization device 102 may include two anchor members 104 and 106 that are coupled via the sliding member 108.

In the present example, the anchor member 104 may include an adjustable anchor portion 202 and a dynamic portion 204 joined by a middle portion 206. While the middle portion 206 is illustrated as connecting to the adjustable anchor portion 202 and dynamic portion 204 at substantially ninety degree angles in the present embodiment, it is understood that other angles may be used. Furthermore, it is understood that a distance D1 representing a distance (relative to the positioning illustrated in FIG. 2) between the adjustable anchor portion 202 and dynamic portion 204 may be varied from that shown.

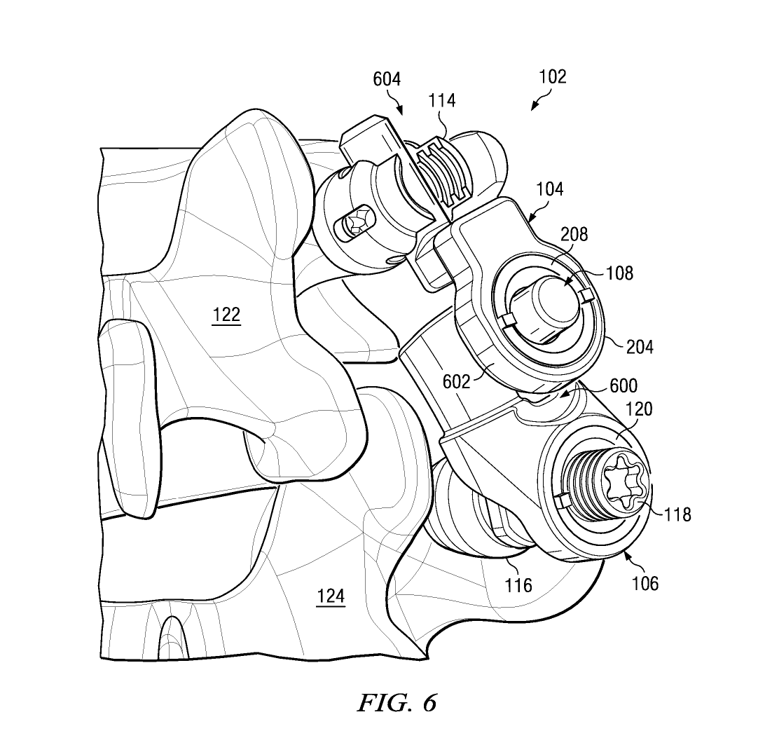

The adjustable anchor portion 202 of the anchor member 104 may be sized to enter a slot (604 of FIG. 6) in the polyaxial head 114. As will be described later, the adjustable anchor portion 202 may be moved within the polyaxial head 114 until a desired position is attained and then locked into place. Accordingly, a distance D2 representing a distance between the polyaxial head 114 and the middle portion 206 may be varied as a position of the adjustable anchor portion 202 varies with respect to the polyaxial head.

The dynamic portion 204 of the anchor member 104 may include an opening containing a threaded or non-threaded bearing element 208 coupled (e.g., welded) to a bearing element 210. The bearing element 210 may serve to retain the bearing element 208 in the opening. The bearing element 208 may include a bore sized to receive a portion of the sliding element 108. In the present example, the bearing element 208 may be sized to allow the sliding element 108 to rotate and slide within the bearing element's bore, enabling the anchor member 104 to move relative to the sliding member 108.

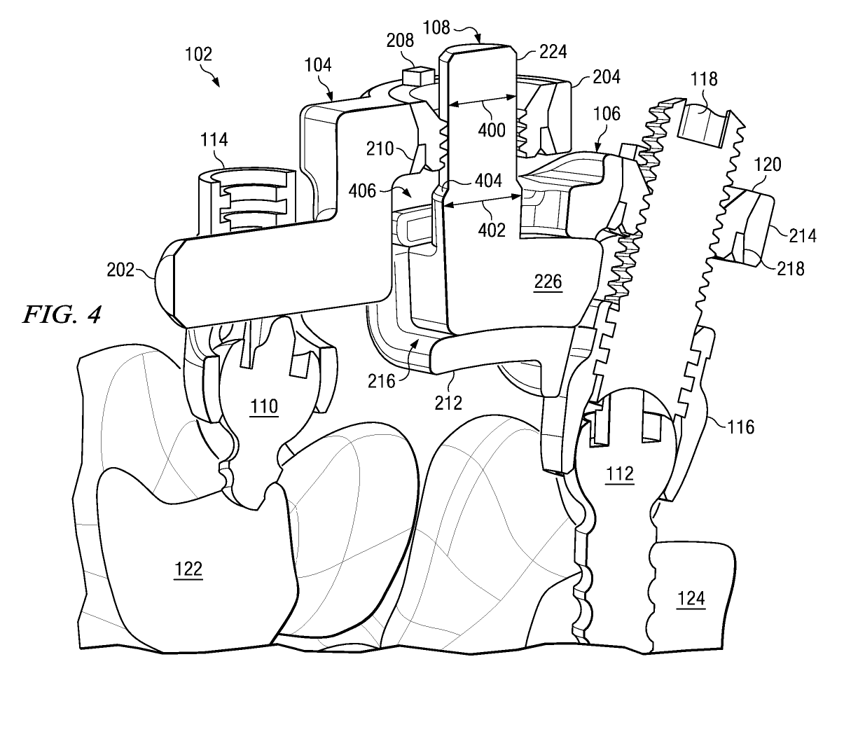

The anchor member 106 may include a cavity portion 212 and an adjustable anchor portion 214. The cavity portion 212 may include a cavity 216 running substantially along a longitudinal axis of the cavity portion, and the cavity may be sized to receive a portion of the sliding member 108. As will be described below, an upper part of the cavity portion 212 (e.g., facing the underside of the dynamic portion 204 of the anchor member 104) may include an opening (406 of FIG. 4) to allow the sliding member 108 to move within the cavity 216.

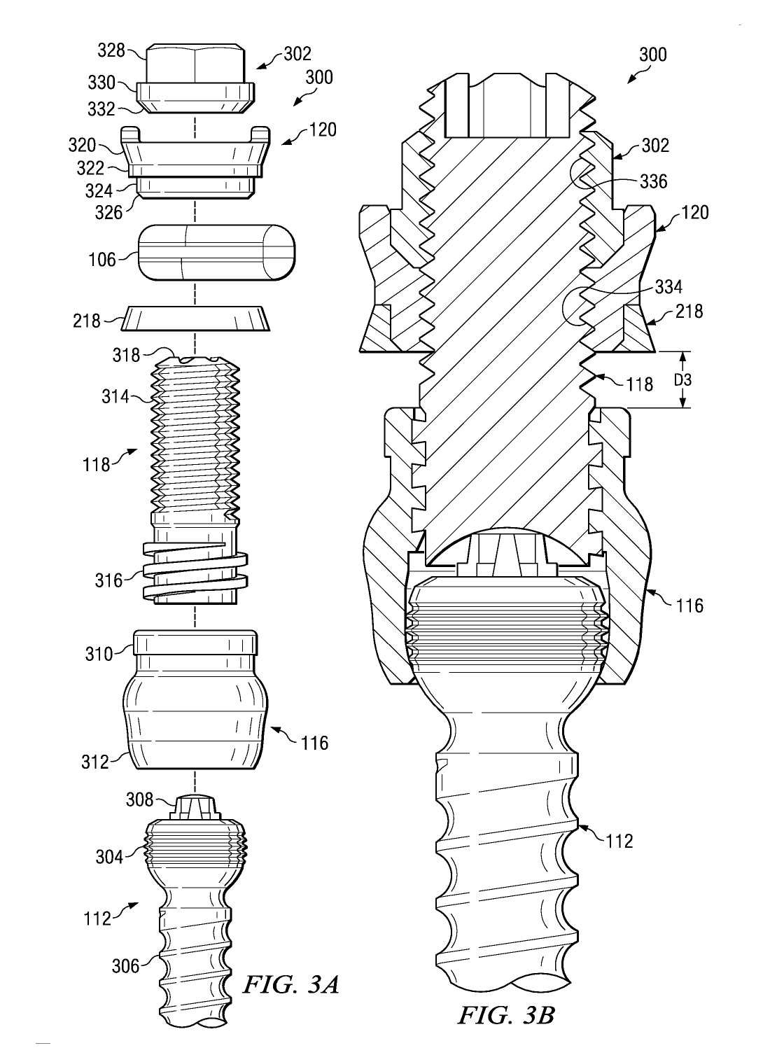

The adjustable anchor portion 214 may include an opening containing the threaded bearing element 120 coupled (e.g., welded) to a bearing element 218. The bearing element 218 may serve to retain the threaded bearing element 120 in the opening. The threaded bearing element 120 may include internal threads 220 configured to engage external threads 222 of the bearing post 118. A locking cap (302 of FIG. 3A) may be used to lock a position of the anchor member 106 relative to the bearing post 118 at a variable distance D3 between the adjustable anchor portion 214 and the polyaxial head 116.

With additional reference to FIG. 3A, one embodiment of a locking assembly 300 that may be used to couple the anchor member 106 to the bone anchor 112 is illustrated in greater detail in a cross-sectional view. The locking assembly may include the bone anchor 110 (e.g., a pedicle screw), polyaxial head 116, bearing post 118, threaded bearing element 120, bearing element 218, and locking cap 302.

The bone anchor 112 may include a proximal portion 304 and a distal portion 306. In the present example, the proximal portion 304 may include a reverse thread that engages a compatible thread form within the polyaxial head 116. When coupled, the polyaxial head 116 may move in relation to the bone anchor 112. The bone anchor 112 may further include an engagement portion 308.

The polyaxial head 116 may include a proximal portion 310 and a distal portion 312, both of which may be threaded. The proximal portion 310 may include a thread form different from that of the distal portion 312. In the present example, the distal portion 312 may be threaded to receive the reverse thread of the proximal portion 304 of the bone anchor 112. The proximal portion 310 may be threaded to receive a portion of the bearing post 118. The threads of the proximal portion 310 may be designed with anti-splay characteristics. For example, the threads may be grooved to accept a dovetail shaped thread. In some embodiments, the proximal portion 310 may be reverse threaded.

The bearing post 118 may include a proximal portion 314 and a distal portion 316, both of which may be threaded. The proximal portion 314 may include a thread form different from that of the distal portion 316. In the present example, the distal portion 316 may include a thread form configured to engage the thread form of the proximal portion 310 of the polyaxial head 116. Although the thread form is not reverse threaded in the present embodiment, it is understood that it may be reverse threaded in other embodiments. The proximal portion 314 may be threaded to engage the threaded bearing element 120 and locking cap 302. The proximal end of the bearing post 118 may include one or more features 318. Such features 318 may, for example, be used to engage a tool for rotating the bearing post 118.

The threaded bearing element 120 may include internal threads (334 of FIG. 3B) configured to engage the proximal portion 314 of the bearing post 118. In the present example, the threaded bearing element 120 may have an exterior surface of varying diameters, including a proximal portion 320, a first intermediate portion 322, a second intermediate portion 324, and a distal portion 326. As will be illustrated in FIG. 3b, the distal portion 326 and second intermediate portion 324 may abut the bearing element 122 and the proximal portion 320 and first intermediate portion 322 may abut the anchor member 106. As the exterior surface of the threaded bearing element 120 may be non-threaded, the anchor member 106 may rotate around the threaded bearing element.

The locking cap 302 may include internal threads (336 of FIG. 3B) configured to engage the proximal portion 314 of the bearing post 118. In the present example, the locking cap 302 may have an exterior surface of varying diameters, including a proximal portion 328, an intermediate portion 330, and a distal portion 332. As will be illustrated in FIG. 3B, the intermediate portion 330 and distal portion 332 may abut an interior surface of the threaded bearing element 120 and the proximal portion 328 may provide a surface for engaging a tool used to tighten the locking cap 302.

With additional reference to FIG. 3B, one embodiment of the locking assembly 300 of FIG. 3A is illustrated in an assembled form. As stated previously, the polyaxial head 116 may generally move relative to the bone anchor 112. However, once the polyaxial head 116 is positioned as desired with respect to the bone anchor 112, it may be desirable to lock the polyaxial head into position. Accordingly, the bearing post 118 may be inserted into the polyaxial head 116 so that the threads of the distal portion 316 of the bearing post engage the threads of the proximal portion 310 of the polyaxial head. The bearing post 118 may then be tightened until the distal end (which may be concave in the present example) contacts the engagement portion 308 of the bone anchor 112. This locks the position of the polyaxial head 116 relative to the bone anchor 112.

As can be seen in FIG. 3B, the threaded bearing element 120 may not contact the polyaxial head 116. More specifically, the position of the threaded bearing element 120 may be adjusted along a longitudinal axis of the bearing post 118 to vary the distance D3 that exists between the threaded bearing element and the polyaxial head 116. This enables a height of the anchor member 106 relative to the polyaxial head 116 to be varied and allows for adjustments to be made to the dynamic stabilization device 102.

The locking cap 302 may be rotated along the longitudinal axis of the bearing post 118 to the desired position and tightened against the threaded bearing element 120. As illustrated, intermediate portion 330 and distal portion 332 of the exterior surface of the locking cap 302 may enter a bore of the threaded bearing element 120 and lock against an internal surface of the threaded bearing element. This may lock the threaded bearing element 120 into place relative to the polyaxial head 116 and may maintain the distance D3 as set.

Referring again to FIG. 2, the sliding portion 108 may include a first portion 224 that extends into the dynamic portion 204 of the anchor member 104 and a second portion 226 that extends into the cavity portion 212 of the anchor member 106. The first portion 224 may be configured with a length D4 that may fit within the bearing element 208, while the second portion 226 may be configured with a length D5 that may fit within the cavity 216. In the present example, the first and second portions 224 and 226 form a substantially ninety degree angle, but it is understood that other angles may be used.

The first and second portions 224 and 226 may be captured within the dynamic portion 204 and cavity portion 212 by the positioning of the anchor members 104 and 106 and/or by other means. For example, a maximum change of position between the vertebral bodies 122 and 124 along a longitudinal axis of the portion 224 may be less than the length D4. Similarly, a maximum change of position between the vertebral bodies 122 and 124 along a longitudinal axis of the portion 226 may be less than the length D5.

In some embodiments, additional means (e.g., a retaining ring, retaining pin, or elastic sleeve) may be provided to capture the first portion 224 and/or second portion 226 within the dynamic portion 204 and cavity portion 212, respectively.

With additional reference to FIG. 4, another cross-sectional view of the dynamic stabilization device 102 illustrates the sliding member 108 in greater detail. As can be seen, in the present embodiment, the portion 224 of the sliding member 108 may have a first diameter represented by arrow 400 and a second diameter represented by arrow 402. The first diameter 400, which is sized to fit within the bearing element 208, may be smaller than the second diameter 402, which is larger than the bore of the bearing element. Accordingly, the diameter 402 may prevent the dynamic portion 204 from contacting the cavity portion 212. A sloped neck 404 may join the two diameters. A slot 406 may be sized to enable movement of the portion 224 along a longitudinal axis of the cavity portion 212.

It is understood that the illustrated cross-sections may be varied. For example, as shown in FIG. 4, the portion 224 is substantially cylindrical and the portion 226 is substantially rectangular. Similarly, the adjustable anchor portion 202 is substantially cylindrical. However, these cross-sectional shapes are for purposes of example only and other shapes may be used. Furthermore, various features (e.g., grooves and/or protrusions) may be provided on the surface of the adjustable anchor portion and/or other components.

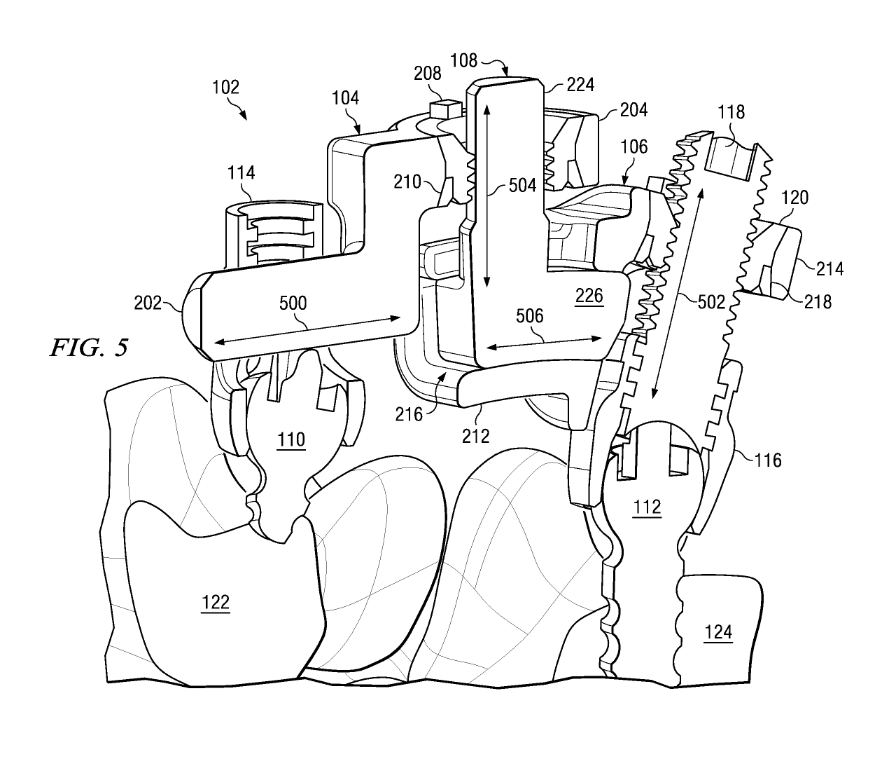

Referring to FIG. 5, while some portions of the dynamic stabilization device 102 may be locked into place after positioning, while other portions may move within a defined range even after positioning. For example, during insertion of the dynamic stabilization device 102, the adjustable anchor portion 202 may be inserted into the polyaxial head 114. Adjustment of the anchor member 104 may then occur along a longitudinal axis (represented by arrow 500) of the adjustable anchor portion 202. Once correctly positioned, a locking nut or other locking means configured to engage threads within the polyaxial head 114 may be tightened. The tightening may lock the adjustable anchor portion 202 into place within the polyaxial head 114. Accordingly, varying distances between the vertebral bodies 122 and 124 may be accounted for during the implantation procedure using the adjustable anchor portion 202. As illustrated, the tightening may also force the adjustable anchor portion 202 against the bone anchor 110, preventing movement between the bone anchor and the polyaxial head 114. In other embodiments, the bone anchor 110 and polyaxial head 114 may be locked into place prior to locking the adjustable anchor portion 202 into place.

Similarly, during insertion of the dynamic stabilization device 102, the adjustable anchor portion 214 may be positioned as desired along a longitudinal axis (represented by arrow 502) of the bearing post 118. Once correctly positioned, the adjustable anchor portion 214 may be locked into placed with respect to the polyaxial head 116 using the locking cap 302 (FIG. 3B), preventing further movement along the longitudinal axis 502. Accordingly, the anchor portion 104 may be locked into position relative to the bone anchor 110 and the anchor portion 106 may be locked into position relative to the bone anchor 112. As described previously, the adjustable anchor portion 214 of the anchor member 106 may still be able to rotate around the longitudinal axis 502.

Even after movement along the longitudinal axes 500 and 502 is stopped, movement may occur between the components of the dynamic stabilization device 102. For example, although the anchor portions 104 and 106 may be locked into position relative to their respective bone anchors 110 and 112, they may still move with respect to one another due to the sliding member 108. For example, the anchor members 104 and 106 may move with respect to one another in a first direction along a longitudinal axis (represented by arrow 504) of the portion 224 as the portion 224 moves within the bearing element 208. The anchor member 104 may also rotate at least partially around the longitudinal axis 504.

Similarly, the anchor members 104 and 106 may move with respect to one another in a second direction along a longitudinal axis (represented by arrow 506) of the portion 226 as the portion 226 moves within the cavity 216. It is understood that the longitudinal axis 506 (and the other longitudinal axes) may actually be curved, and so the movement may be along a curved path rather than a straight line. Accordingly, the anchor member 104 may rotate and slide with respect to the anchor member 106 within the range provided by the sliding member 108, and the anchor member 106 may rotate with respect to the bearing post 118. As discussed above, such movement may be limited. It is understood that such movement may occur simultaneously or separately (e.g., rotation around and/or movement may occur around one or both axes 502 and 504, and/or along one or both axes 504 and 506).

Referring to FIG. 6, a perspective view of one embodiment of the dynamic stabilization device 102 of FIG. 1 is illustrated. As discussed previously, the sliding member 108 may move with respect to the anchor member 106. In the present example, the anchor member 106 may include an indentation 600 having a curved profile that substantially matches a curved outer surface 602 of the dynamic portion 204 of the anchor member 104. Accordingly, the anchor member 104 may move towards the anchor member 106 until the outer surface 602 contacts the indentation 600. It is noted that, due to the substantially similar curves of the outer surface 602 and indentation 600, the anchor member 104 may rotate around the sliding member 108 even when in contact with the anchor member 106.

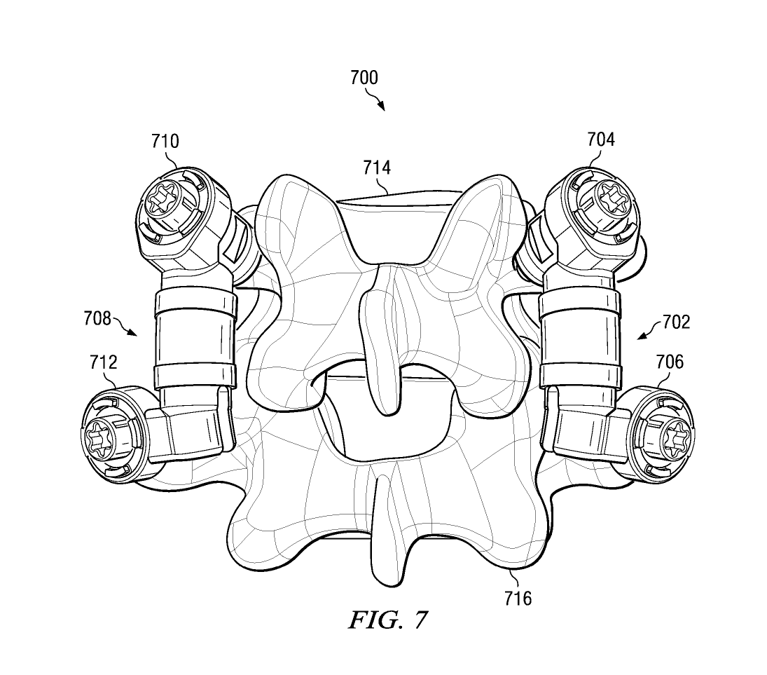

Referring to FIG. 7, another embodiment of a dynamic stabilization system 700 is provided. In the present example, the dynamic stabilization system 700 includes two dynamic stabilization devices 702 and 708. The dynamic stabilization device 702 may include an upper member 704 and a lower member 706, at least a portion of which may be offset. The dynamic stabilization device 708 may include an upper member 710 and a lower member 712, at least a portion of which may be offset. The offset portions of the lower members 706 and 712 may, for example, minimize the vertical distance needed for the dynamic stabilization devices 702 and 708.

As illustrated, the upper portions 704 and 710 of the dynamic stabilization devices 702 and 708 may be coupled to a vertebral body 714 and the lower portions 706 and 712 of the dynamic stabilization devices may be coupled to a vertebral body 716. A center of rotation (not shown) may be defined between the vertebral bodies 714 and 716, and the dynamic stabilization devices 702 and 708 may restrict motion to a spherical shell or other three dimensional shape around the center of rotation. Accordingly, portions of the dynamic stabilization devices 702 and 708 may be aligned with the center of rotation.

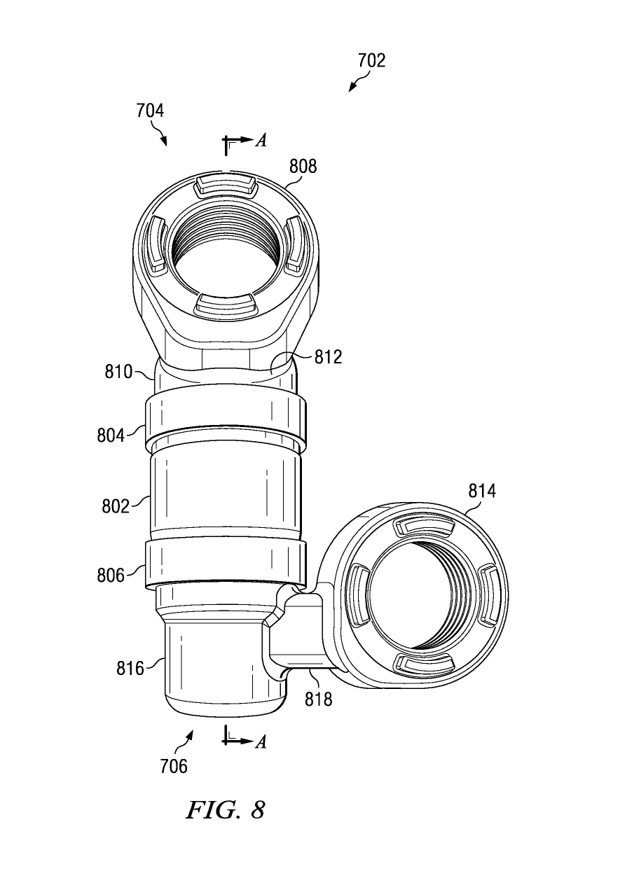

Referring to FIG. 8, one embodiment of the dynamic stabilization device 702 of FIG. 7 is illustrated. The dynamic stabilization device 702 may include the upper and lower members 704 and 706, respectively, which may slidingly engage each other. In the present example, a cover 802 may be coupled to the upper member 704 by a cover attachment band 804 and to the lower member 706 by a cover attachment band 806.

The upper member 704 may include an anchor portion 808 and a sliding portion 810. A stem 812 may join the anchor portion 808 and sliding portion 810. It is understood that the anchor portion 808 may be coupled to the sliding portion 810 at a variety of angles and the stem 812 may be any desired length.

The lower member 706 may include an anchor portion 814 and a sliding portion 816. The anchor portion 814 may be permanently coupled (e.g., welded) to the sliding portion 816. It is understood that the anchor portion 814 may be coupled to the sliding portion 816 at a variety of different angles and a stem 818 of the anchor portion 814 may be any desired length. This offset may, for example, enable the dynamic stabilization device 702 to be positioned in a smaller space (with respect to a length of the device).

Referring to FIG. 9, a side view of the dynamic stabilization device 702 of FIG. 8 is illustrated along lines A-A. As will be described later in greater detail, a stop pin 902 may be provided to prevent movement beyond defined parameters.

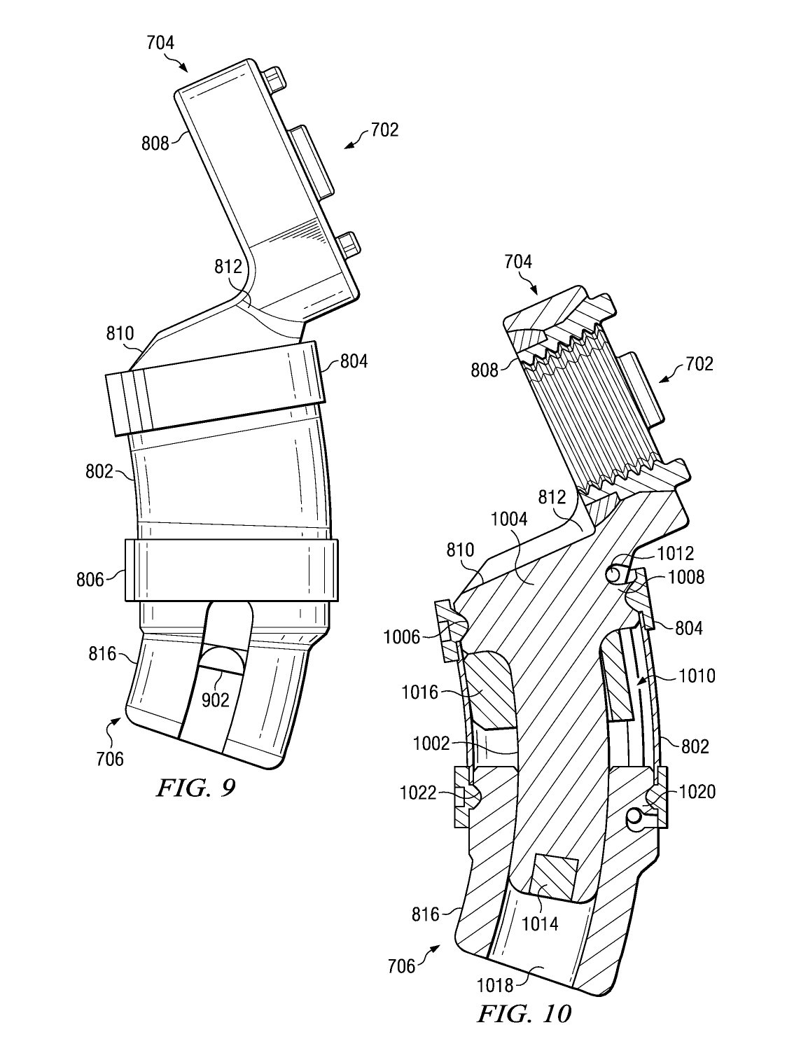

With additional reference to FIG. 10, a cross-sectional view of one embodiment of the dynamic stabilization device of FIG. 9 is illustrated. As can be seen, the sliding portion 808 of upper member 704 may include a shaft 1002. The shaft 1002 may be coupled to a neck 1004 that may be wider than the shaft 1002 and may be coupled to the stem 812. The neck 1004 may include a surface feature 1006 (e.g., a groove, bump or other feature) configured to receive or otherwise engage the upper attachment band 804. It is understood that the surface feature 1006 may not be located on the neck 1004, but may be positioned elsewhere on the upper member 704. A corresponding surface feature 1020 may be present on the lower member 706.

The upper member 704 may also include a feature 1008 for engaging a tension mechanism 1010 (e.g., a tension band). In the present example, the feature 1008 may be a cleat or other extension, but it is understood that the tension mechanism 1010 may be coupled to the upper member 704 in many different ways. As illustrated, a groove 1012 may be formed at least partially around the feature 1008 for receiving the tension mechanism 1010. A corresponding groove 1022 may be present on the lower member 706.

A stop mechanism 1014 (e.g., the stop pin 902) may prevent movement of a distal end (relative to the anchor portion 808) of the shaft 1002 passed a defined point with respect to the lower member 706. The sliding portion 816 of the lower member 706 may include an opening 1018 configured to receive the shaft 1002.

An extension bumper 1016 may be positioned along the shaft 1002 between the neck 1004 and the sliding portion 816. The extension bumper 1016 may prevent the neck 1004 from contacting the sliding portion 816 and may provide a cushion to prevent a hard stop when the dynamic stabilization device 702 is in a fully compressed state. Accordingly, varying the height of the extension bumper 1016, as well as its material properties, may vary the amount of movement between the neck sliding portions 810 and 816 and/or the amount of cushioning provided by the extension bumper.

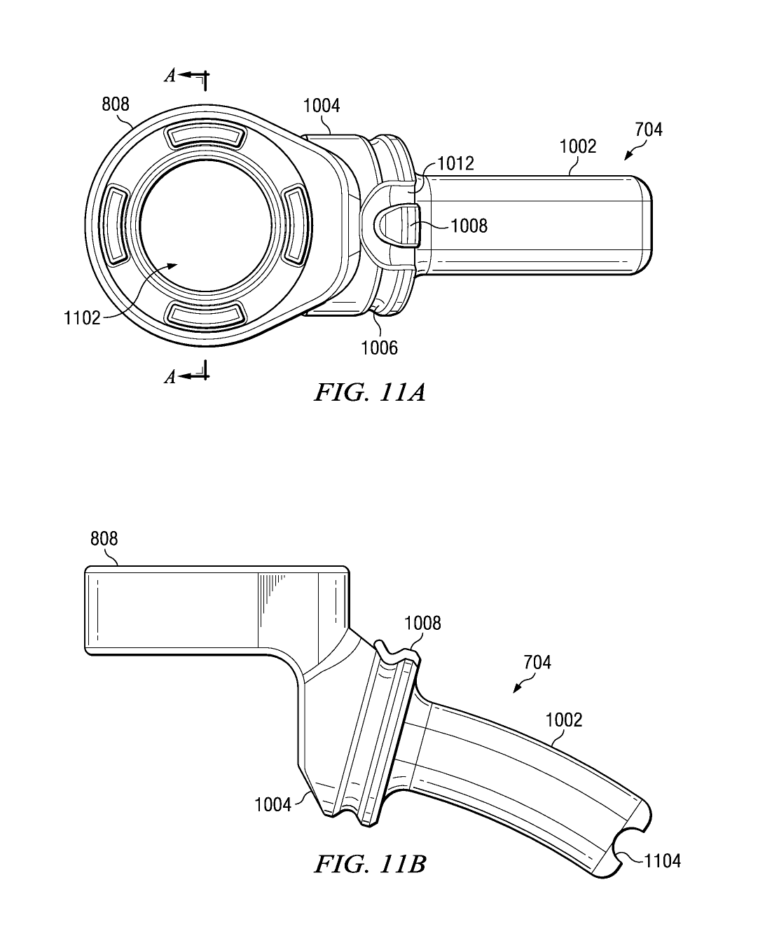

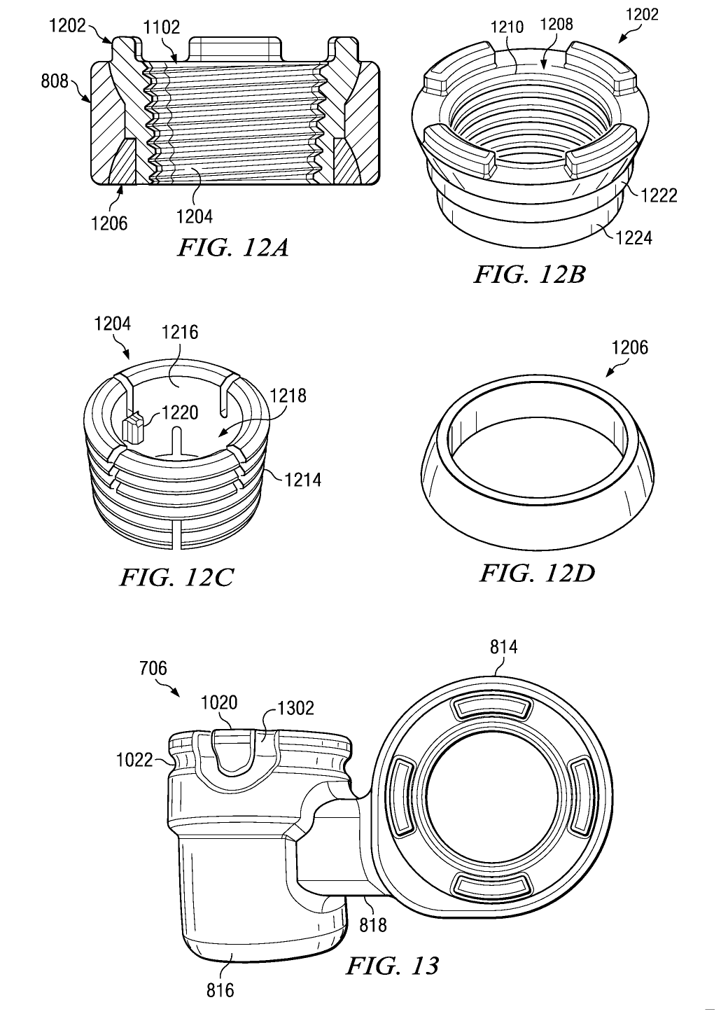

Referring to FIGS. 11A and 11B, a top view and side view, respectively, of one embodiment of the upper member 704 are illustrated. In the present example, the anchor portion 808 includes a bore 1102 (FIG. 11A) configured to receive a bearing element (FIG. 12A). The surfaces of the bore 1102 may be smooth to enable the bearing element to rotate within the bore 1102 or may include one or more surface features to engage the bearing element and minimize or eliminate movement of the bearing element relative to the bore 1102.

The shaft 1002 may have a relatively square cross-section having rounded corners, although any shape of cross-section may be used. In the present example, the shaft 1002 may be curved (as illustrated in FIG. 11B) along a path from the neck 1004 away from the anchor portion 808. The curve may match a curve of the opening 1018 (FIG. 10) and may be designed to maintain movement of the dynamic stabilization device 704 around the center of rotation. The distal end (relative to the anchor portion 808) of the shaft 1002 may include a groove 1104 or other engaging feature for engaging the stop pin 914.

The groove 1012 formed in the neck 1004 may extend at least partially around the cleat 1008. The groove 1012 may be sized to receive the tension band 1010 (FIG. 10) so that the tension band is substantially level with the surface of the neck 1004. This may enable the cover attachment band 804 (FIG. 8) to fasten to the neck 1004 without interfering with the tension band 1010.

Referring to FIG. 12A, a more detailed cross-section of the anchor portion 808 of upper member 704 taken along lines A-A of FIG. 11A is provided. As illustrated in FIG. 12A, the bore 1102 may include a tapered bearing element 1202 coupled to a bushing ring 1206 and containing a collet 1204.

With additional reference to FIGS. 12B-12D, the bearing element 1202 (FIG. 12B) may include a bore 1208 having a partially or totally threaded inner surface 1210. The collet 1204 (FIG. 12C), which may be tapered, may have an external threaded surface 1214 configured to engage the threaded surface 1210. An interior surface 1216 of a bore 1218 of the collet 1204 may include one or more protrusions 1220. As will be described later in greater detail, the protrusion 1220 may engage one or more grooves in a bearing post (FIG. 17).

The bearing element 1202 may also include a tiered or multi-level outer surface 1222 configured to abut the surface of the bore 1102. In the present example, the outer surface 1222 may include an indentation 1224 configured to receive the bushing ring 1206 (FIG. 12D). The bushing ring 1206 may secure the bearing element 1202 to the anchor portion 808. For example, the bearing element 1202 may be inserted into the bore 1102 of the anchor portion 808, and the bushing ring 1206 may be secured (e.g., welded) to the bearing element to retain the bearing element within the bore 1102 while still allowing rotation of the bearing element within the bore.

Referring to FIG. 13, a top view of one embodiment of the lower member 706 of FIG. 7 is illustrated. In the present example, the anchor portion 814 may be offset from the sliding portion 816. Such an offset may, for example, minimize an amount of vertical space (e.g., from the anchor member 808 to the anchor member 814) needed for the dynamic stabilization device 702.

The anchor portion 814 may include a bearing element, collet, and bushing ring similar or identical to those described with respect to FIGS. 12A-12D for the anchor portion 808. Accordingly, the anchor portion 814 is not described in detail herein.

The sliding portion 816 may include the bore 1102 (not shown) for receiving the shaft 1002 of the upper member 704. The sliding portion 816 may include the feature 1020 for engaging the tension band 1010. In the present example, the feature 1020 may be a cleat or other extension, but it is understood that the tension mechanism 1010 may be coupled to the lower member 706 in many different ways. As illustrated, a groove 1302 may be formed at least partially around the feature 1020 for receiving the tension mechanism 1010.

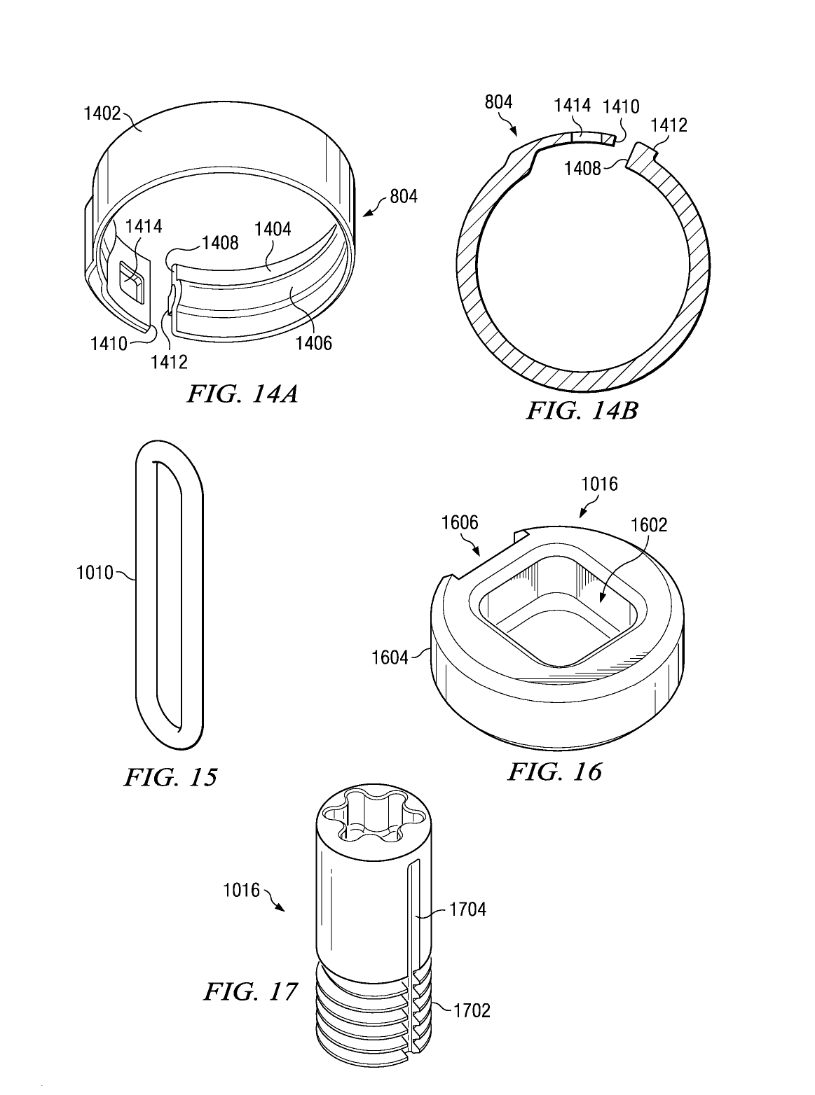

Referring to FIGS. 14A and 14B, one embodiment of the cover attachment band 804 of FIG. 8 is illustrated in greater detail. The cover attachment band 806 may be substantially similar or identical to the cover attachment band 804 and is not described in detail herein. In the present example, the cover attachment band 804 may have a substantially ring-like shape having a closable opening in the ring. The cover attachment band 804 may have a substantially smooth outer surface 1402. An inner surface 1404 may include a protrusion 1406 for engaging the groove 1006 (FIG. 10) of the upper member 704. It is understood that the groove 1006 and protrusion 1406 may be switched (e.g., the groove may be located on the cover attachment band 804 and the protrusion may be located on the upper member 704). Alternate or additional means may also be used to maintain a desired position of the cover attachment band 804 relative to the upper member 704.

In the present example, the substantially ring-like shape of the cover attachment band 804 may include a first end 1408 and a second end 1410. The cover attachment band 804 may include a locking means for coupling the first and second ends 1408 and 1410. For example, the first end 1408 may include a protrusion 1412 and the second end 1410 may include a matching opening 1414 designed to receive the protrusion.

Referring to FIG. 15, one embodiment of the tension band 1010 (FIG. 10) is illustrated. The tension band 1010 may be formed from an elastomeric material and may resist flexion of the dynamic stabilization device 702. In the present example, the tension band 1010 may be neutral (i.e., exerting no force) when the vertebral bodies 714 and 716 of FIG. 7 are in a neutral position. However, it is understood that the tension band 1010 may be configured to provide tension for different positions of the vertebral bodies 714 and 716.

In some embodiments, multiple tension bands may be provided for use with the dynamic stabilization device 702. For example, the tension bands may be provided in a kit for use by a surgeon. The tension bands may have different configurations (e.g., lengths, cross-sectional shapes, and/or materials) and one or more of the tension bands may be selected for use with the dynamic stabilization device 702 based on the particular patient. For example, if a surgeon wants the dynamic stabilization device 702 to permit less flexion, then the surgeon may select a relatively short tension band. Alternatively, if the surgeon wants the dynamic stabilization device 702 to permit more flexion, then the surgeon may select a longer tension band. Accordingly, various levels of flexion may be controlled by altering the length of the tension band. The tension band may also be selected to permit varying amounts of slackness. In some embodiments, one or more tension bands may be used simultaneously.

The tension bands may also have different material compositions to enable a surgeon to select a tension band with desired characteristics. For example, the surgeon may select a tension band made of a relatively inelastic material to provide a relatively hard stop when the outer limit of flexion is reached, or may select a tension band with a relatively elastic material to provide a dampening effect that provides increasing resistance to the flexion movement until the outer limit of flexion is reached.

Referring to FIG. 16, one embodiment of the extension bumper 1016 is illustrated. The extension bumper 1016 may include a bore 1602 that receives the shaft 1002 (FIG. 10). For example, if the shaft 1002 has a substantially square cross-section, the bore 1602 may also have a substantially square cross-section. This may prevent the shaft 1002 from rotating within the bore 1602. It is understood, however, that the cross-sectional shape of the bore 1602 may not correspond to the cross-sectional shape of the shaft 1002 in some embodiments. An outer surface 1604 of the extension bumper 1016 may be substantially smooth. The extension bumper 1016 may be coupled to the upper member 704, shaft 1002, or to one or more other components of the dynamic stabilization device 702, or may not be coupled at all.

A groove 1606 may be formed in the outer surface 1604 to receive the tension band 1010. The groove 1606 may, for example, prevent the tension band 1010 from exerting constant pressure on the extension bumper 1016. Such pressure may deform the extension bumper 1016 and may also result in an alteration of the tension in the tension band 1010 if the tension band begins to deform the extension bumper 1016. In the present example, the height of the extension bumper 1016 may vary from a first height on the side containing the groove 1606 to a second height on the opposite side. The first height may be greater than the second height to configure the extension buffer 1016 with respect to the curvature of the shaft 1002, as illustrated in FIG. 10.

In the present example, the extension bumper 1010 may be formed from an elastomeric material, but it is understood that it may be formed from any suitable material or combination of materials. When the vertebral bodies 714 and 716 are in extension (e.g., when a person bends backwards), the extension bumper 1016 may compress within the dynamic stabilization device 702 and resist further extension. Accordingly, the extension bumper 1016 may provide a dampening effect until fully compressed, at which time no further extension may be possible.

In some embodiments, multiple extension bumpers may be provided for use with the dynamic stabilization device 702. For example, the extension bumpers may be provided in a kit (alone or with tension bands) for use by a surgeon. The extension bumpers may have different configurations (e.g., thicknesses, cross-sectional shapes, and/or materials) and one or more of the extension bumpers may be selected for use with the dynamic stabilization device 702 based on the particular patient. For example, if a surgeon wants the dynamic stabilization device 702 to permit less extension, then the surgeon may select a relatively thick (i.e., long) extension bumper. Alternatively, if the surgeon wants the dynamic stabilization device 702 to permit more extension, then the surgeon may select a narrower (i.e., shorter) extension bumper. Accordingly, various levels of extension may be controlled by altering the length of the extension bumper. In some embodiments, one or more of the extension bumpers may be stackable to allow for the use of multiple extension bumpers simultaneously.

The extension bumpers may also have different material compositions to enable a surgeon to select an extension bumper with desired characteristics. For example, the surgeon may select an extension bumper made of a relatively rigid material to provide a relatively hard stop when the outer limit of extension is reached, or may select an extension bumper with a relatively elastic material to provide a dampening effect that provides increasing resistance to the extension movement until the outer limit of extension is reached.

In the present embodiment, the tension band 1010 and the extension bumper 1016 may not be exerting force at the same time. For example, the tension band 1010 may be neutral (e.g., exerting no force) when the vertebral bodies 714 and 716 are in a neutral position. Similarly, the extension bumper 1016 may only exert force when compressed, which may not happen when the vertebral bodies 714 and 716 are in a neutral position. Accordingly, in such an embodiment, the tension band 1010 may only exert force when the vertebral bodies 714 and 716 are in flexion and the extension bumper 1016 may only exert force when the vertebral bodies are in extension. However, it is understood that the tension band 1010 and extension bumper 1016 may exert force simultaneously in other embodiments.

Referring to FIG. 17, one embodiment of a bearing post 1700 is illustrated. The bearing post 1700 may include threads 1702 for engaging threads in a polyaxial head. In the present example, the bearing post 1700 may include one or more grooves 1704. The groove 1704 may receive the protrusion 1220 (FIG. 12C) of the collet 1204 and may prevent the collet from turning relative to the bearing post 1700 when the bearing element 1202 (FIG. 12A) is rotated relative to the bore 1102.

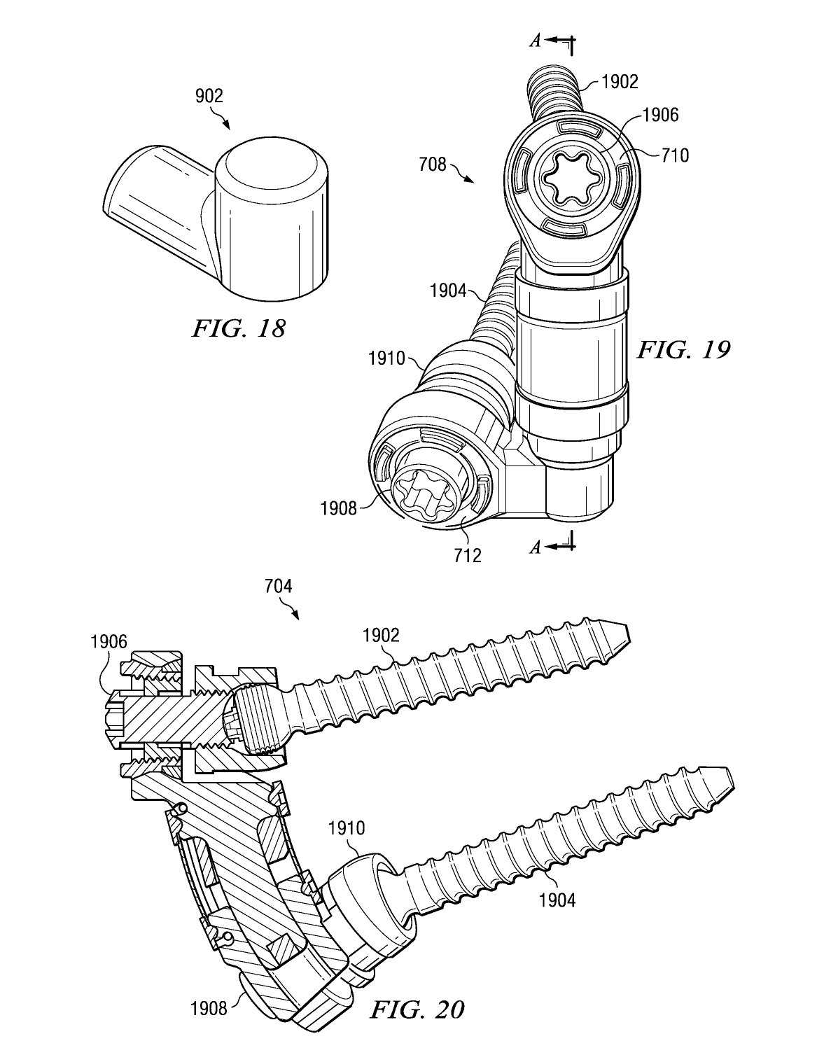

Referring to FIG. 18, an embodiment of the stop pin 902 of FIG. 9 is illustrated.

Referring to FIGS. 19 and 20, an embodiment of the dynamic stabilization device 708 of FIG. 7 is illustrated. As the dynamic stabilization device 708 may be similar or identical to the dynamic stabilization device 702 described above, it is not described in detail herein. It is noted that an offset portion of the lower member 712 of the dynamic stabilization device 708 may be offset in an opposite direction than the offset portion of the lower member 706 of the dynamic stabilization device 702.

Also illustrated are bone anchors 1902 and 1904, upper portions of bearing posts 1906 and 1908, and a portion of a polyaxial head 1910 that may be coupled to bone anchor 1904 and bearing post 1908.

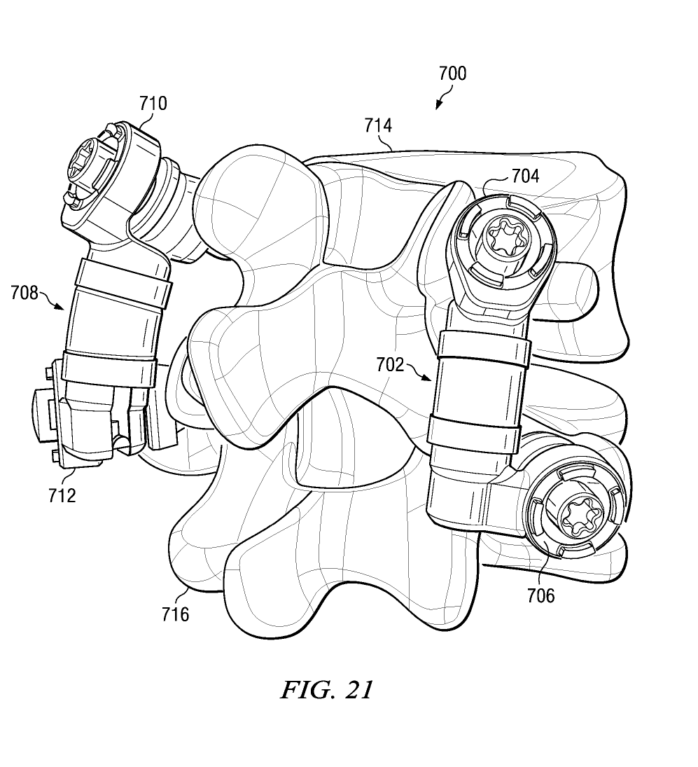

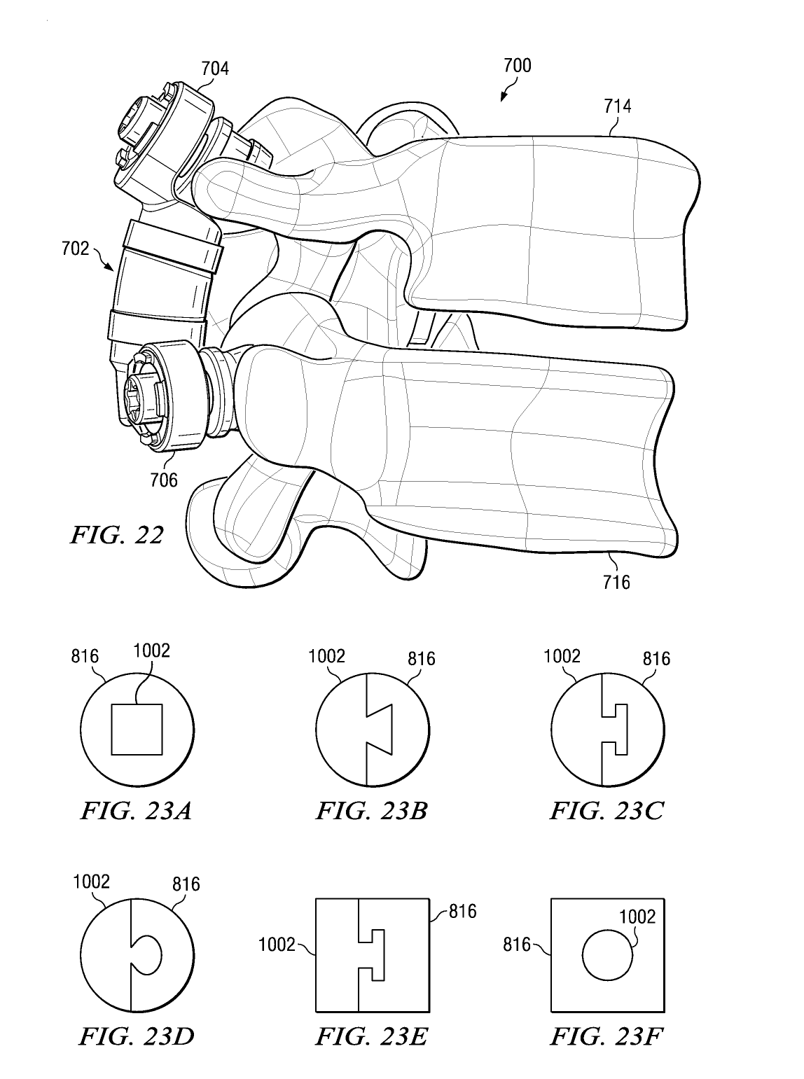

Referring to FIGS. 21 and 22, additional views of the dynamic stabilization system 700 of FIG. 7 are provided.

In operation, bone anchors may be inserted into the vertebral bodies 714 and 716. The polyaxial heads may be coupled to the bone anchors before, during, and/or after the insertion process. A bearing post 1100 may be inserted into each polyaxial head.

The bore 1218 of the collet 1204 may be placed over the bearing post 1700, and the bearing element 1202 may be rotated with respect to the bore 1102. During rotation of the bearing element 1202, the collet 1204 may be prevented from rotating due to the protrusion 1220 extending into the groove 1704 of the bearing post 1700. Accordingly, as the bearing element 1202 is rotated, the collet 1204 is tightened against the bearing post 1700. It is understood that a gap may exist between the bearing element 1202 and the polyaxial head in some embodiments.

Referring to FIGS. 23A-23F, various embodiments of cross-sectional configurations between the shaft 1002 and sliding portion 816 are illustrated. It is understood that these are merely examples, and that many different cross-sectional configurations are possible. In some embodiments, although not shown, the shaft 1002 and sliding portion 816 may be reversed.

In some embodiments, after placement of the dynamic stabilization device 702 on the bone anchors and before locking down the polyaxial heads by tightening the bearing posts, the device may be aligned with a center of rotation. In other embodiments, the polyaxial heads, bearing posts, and/or bores of the anchor members may be aligned with a center of rotation prior to placement of the dynamic stabilization device 702. As described previously, when aligned, the dynamic stabilization devices 702 and 708 may restrict motion to a three dimensional surface centered on the center of rotation. An alignment aid may be used during the alignment process, such as an alignment device described in U.S. patent application Ser. No. 11/467,798 entitled “ALIGNMENT INSTRUMENT FOR DYNAMIC SPINAL STABILIZATION SYSTEMS” and filed on Aug. 28, 2006, which is incorporated herein by reference.

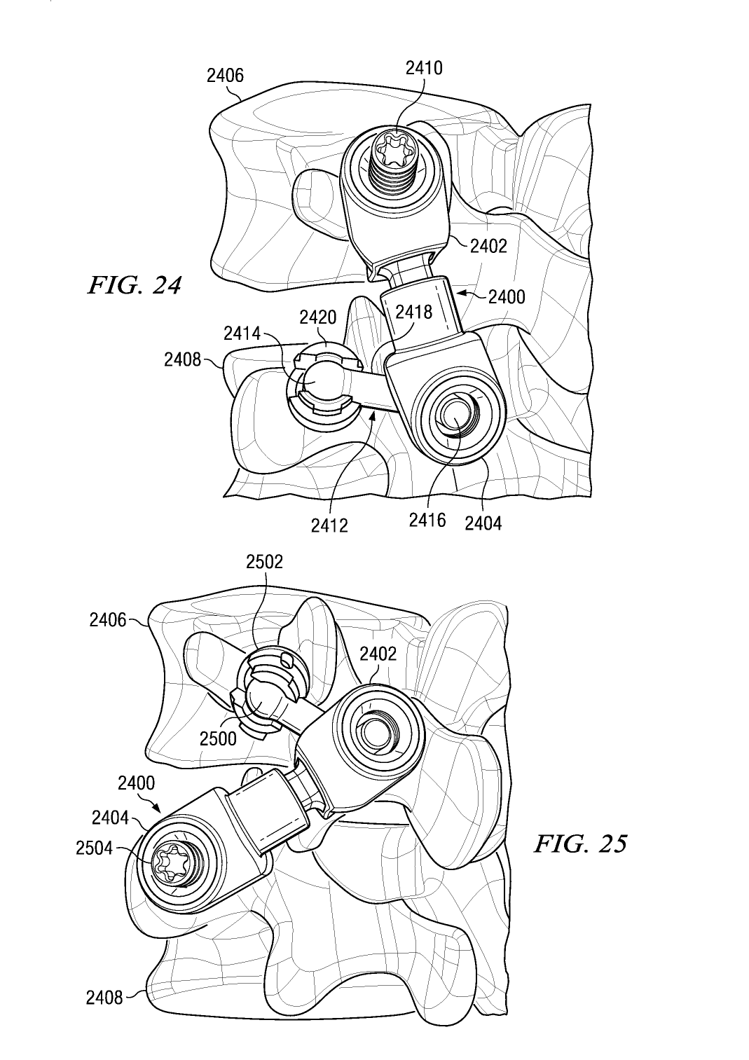

Referring to FIG. 24, in another embodiment, a dynamic stabilization device 2400 is illustrated. Internally, the dynamic stabilization device 2400 may be similar or identical to the dynamic stabilization device 702 of FIG. 7 in that the dynamic stabilization device 2400 may include upper and lower members 2402 and 2404, respectively, which may interact as previously described. For example, the dynamic stabilization device 2400 may include an extension bumper and/or a tension band that may regulate the interaction of the upper and lower members 2402 and 2404 during extension and flexion, respectively. Externally, the dynamic stabilization device 2400 may not include the offset illustrated with the dynamic stabilization device 702. Instead, anchor portions of the upper and lower members 2402 and 2404 may be positioned substantially along a single longitudinal axis (which may be curved).

In the present example, the upper member 2402 may be coupled to a vertebral body 2406 via a bearing post 2410, and the lower member 2404 may be coupled to a vertebral body 2408 via a rod 2412. The bearing post 2410 may be identical or similar to the bearing post 118 of the locking assembly 300 of FIG. 3A. The rod 2412 may include a first end 2414 and a second end 2416. In the present example, the first end 2414 may have a substantially spherical shape (e.g., like a bearing) and the second end 2416 may include a threaded post. The threaded post may be substantially perpendicular to a longitudinal axis of a rod portion 2418 connecting the first and second ends 2414 and 2416. It is understood that the shapes and cross-sectional configurations of the first and second ends 2414 and 2416 and the rod portion 2418, as well as the perpendicular orientation of the second end, are for purposes of example and may be altered to provide a desired configuration.

In the present example, the bearing of the first end 2414 may fit into a polyaxial head 2420. The polyaxial head 2420 may be similar or identical to the polyaxial head 116 of FIG. 3A. The first end 2414 may rotate within the polyaxial head 2420 until secured by a locking cap or other locking mechanism. The threaded post of the second end 2416 may be identical or similar to the bearing post 118 of the locking assembly 300 of FIG. 3A and may be coupled to the lower member 2404 using various locking assembly components, such as those illustrated in FIG. 3A. Accordingly, the rod 2412 may enable the dynamic stabilization device 2400 to be offset from the polyaxial head 2420 without having an offset integrated into the design of the dynamic stabilization device itself. It is understood that the rod 2412 may be used with one or both of the upper and lower members 2402 and 2404, and may be used with a device having an integrated offset (e.g., the dynamic stabilization device 702 of FIG. 7).

Referring to FIG. 25, the dynamic stabilization device 2400 of FIG. 24 is illustrated with the upper member 2402 coupled to the vertebral body 2406 via a rod 2500 and polyaxial head 2502. The lower member 2404 is coupled to the vertebral body 2408 via a bearing post 2504. As the rod 2500, polyaxial head 2502, and bearing post 2504 may be identical or similar to the rod 2412, polyaxial head 2420, and bearing post 2410 of FIG. 24, they are not described further herein.

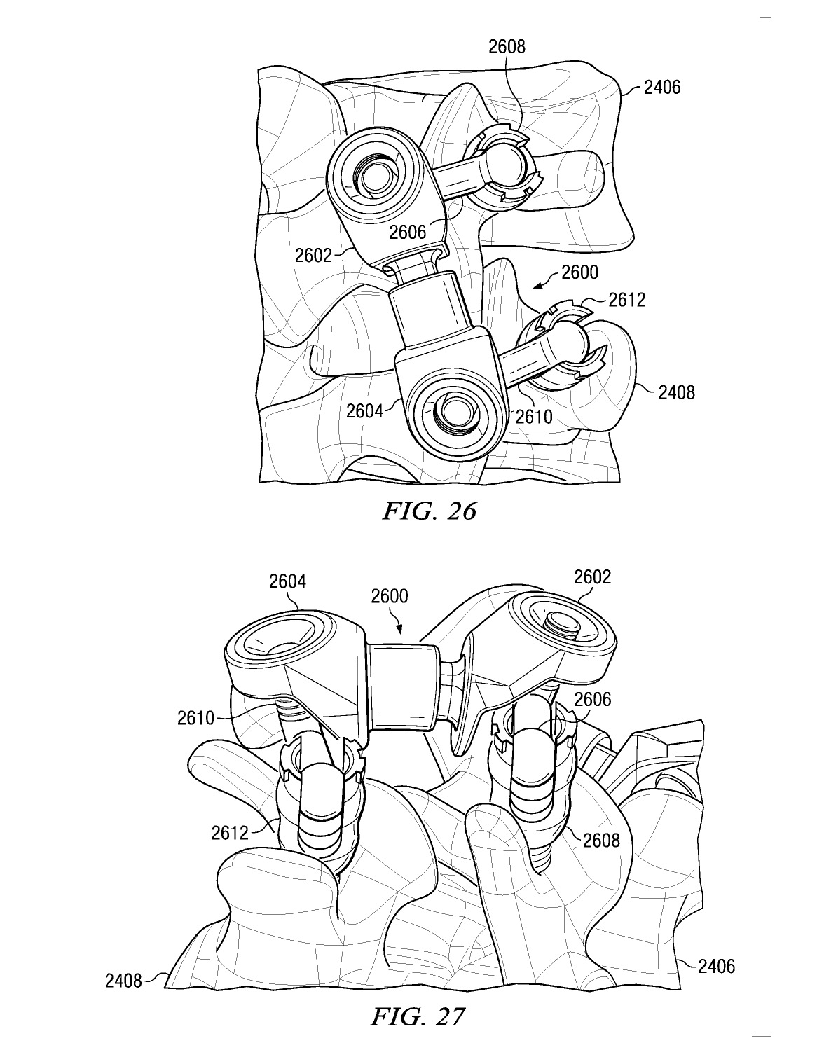

Referring to FIGS. 26 and 27, a dynamic stabilization device 2600 is illustrated with an upper member 2602 coupled to a polyaxial head 2608 by a rod 2606. A lower member 2604 is coupled to a polyaxial head 2612 by a rod 2610. The polyaxial heads 2608 and 2612 may be coupled to vertebral bodies 2406 and 2408, respectively. As the upper member 2602, lower member 2604, rods 2606 and 2610, and polyaxial heads 2608 and 2612 may be similar or identical to the corresponding components described above with respect to FIG. 24, they are not described further herein.

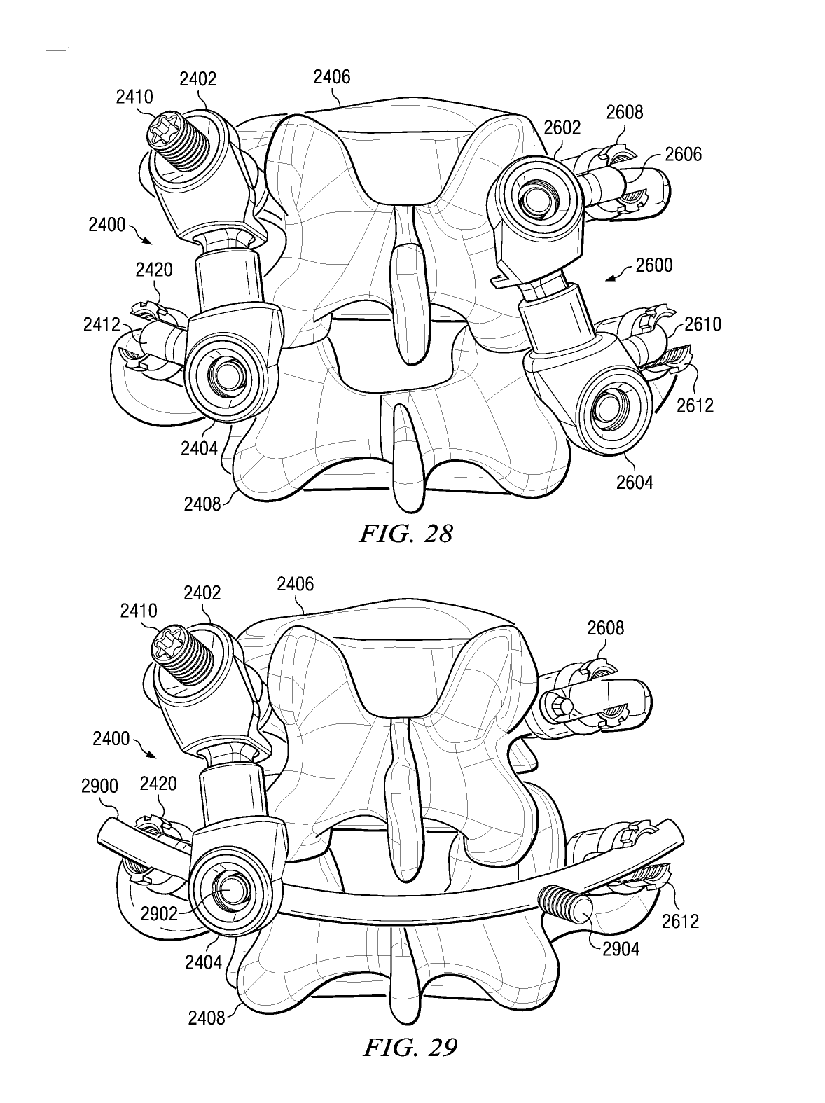

Referring to FIG. 28, the dynamic stabilization device 2400 of FIG. 24 and the dynamic stabilization device 2600 of FIG. 26 are illustrated simultaneously coupled to vertebral bodies 2406 and 2408.

Referring to FIG. 29, the dynamic stabilization device 2400 of FIG. 24 is illustrated with the upper member 2402 coupled to the bearing post 2410. A rod 2900 extends from the polyaxial head 2420 to the polyaxial head 2612. The rod 2900 may include threaded posts 2902 and 2904. The threaded posts 2902 and 2904 may be identical or similar to the bearing post 118 of the locking assembly 300 of FIG. 3A and may be coupled to the lower member 2404 and a lower member of another dynamic stabilization device (not shown) using various locking assembly components, such as those illustrated in FIG. 3A. In the present example, the rod 2900 may be curved, but it is understood that the rod may have various shapes and cross-sections. Furthermore, it is understood that the location of the threaded posts 2902 and 2904 may vary in some embodiments.

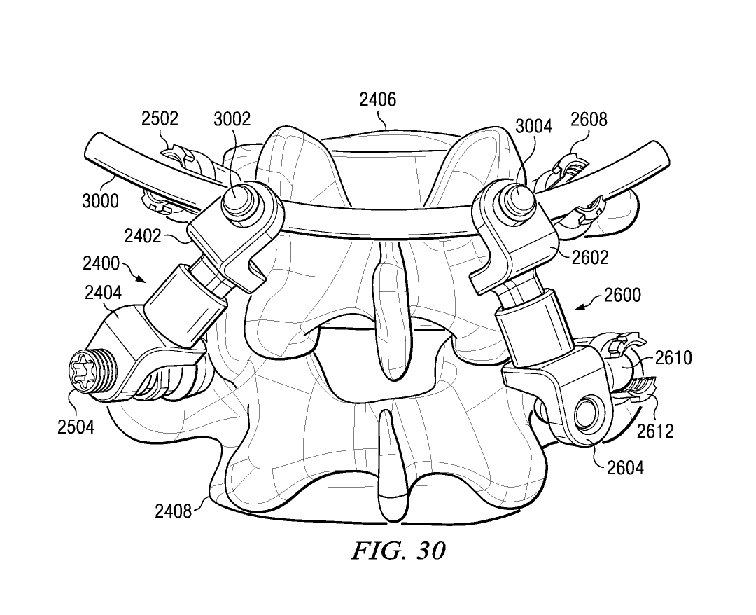

Referring to FIG. 30, the dynamic stabilization device 2400 of FIG. 24 is illustrated with lower member 2404 coupled to a bearing post 2504 (FIG. 25) and upper member 2402 coupled to a rod 3000. The dynamic stabilization device 2600 is illustrated with lower member 2604 coupled to the rod 2610 and upper member 2602 coupled to the rod 3000.

The rod 3000 may extend from the polyaxial head 2502 (FIG. 25) to the polyaxial head 2608 (FIG. 26). The rod 3000 may include threaded posts 3002 and 3004. The threaded posts 3002 and 3004 may be identical or similar to the bearing post 118 of the locking assembly 300 of FIG. 3A and may be coupled to the upper members 2402 and 2602 using various locking assembly components, such as those illustrated in FIG. 3A. In the present example, the rod 3000 may be curved, but it is understood that the rod may have various shapes and cross-sections. Furthermore, it is understood that the location of the threaded posts 2902 and 2904 may vary in some embodiments. Although shown with threaded posts, it is understood that the rods 2900 and 3000 may be coupled to one or more dynamic stabilization devices using other fastening mechanisms (e.g., pins, clamps, screws, and/or dovetails).

Although only a few exemplary embodiments of this disclosure have been described in details above, those skilled in the art will readily appreciate that many modifications are possible in the exemplary embodiments without materially departing from the novel teachings and advantages of this disclosure. Also, features illustrated and discussed above with respect to some embodiments can be combined with features illustrated and discussed above with respect to other embodiments. Accordingly, all such modifications are intended to be included within the scope of this disclosure.

What is claimed is:

1. A dynamic stabilization device having an integrated offset comprising:
a first member having first and second portions aligned along a longitudinal axis, wherein the first portion is configured to rotationally couple to a first polyaxial head and includes a first intersecting axis that extends through the first portion at an angle to the longitudinal axis to intersect a center point; and
a second member having a third portion aligned along the longitudinal axis and slideably engaging the second portion, and a fourth portion offset from the longitudinal axis and configured to rotationally couple to a second polyaxial head, the fourth portion including a second intersecting axis that extends through the fourth portion at an angle to the longitudinal axis to intersect the center point, wherein the longitudinal axis is curved to maintain the intersection of the first and second intersecting axes with the center point as the center point moves along a curved three dimensional surface during movement of the first member relative to the second member.

2. The dynamic stabilization device of claim 1 wherein the fourth portion is offset from the longitudinal axis at a substantially ninety degree angle.

3. The dynamic stabilization device of claim 1 further comprising a neck coupling the third portion to the fourth portion.

4. The dynamic stabilization device of claim 1 wherein the third portion includes a bore oriented along the longitudinal axis and the fourth portion includes a bore oriented substantially perpendicularly to the longitudinal axis.

5. A dynamic stabilization system having an offset member for a single dynamic device comprising:
an offset member having a rod connecting a shaped first portion to a threaded second portion, wherein a first longitudinal axis of the threaded second portion is angled relative to a second longitudinal axis of the rod, and wherein the shaped first portion is configured to couple to a first polyaxial head;
a first dynamic member having first and second portions oriented along a third longitudinal axis, wherein the first portion is configured to rotationally couple to a second polyaxial head and includes a first intersecting axis that extends through the first portion at an angle to the third longitudinal axis to intersect a center point; and
a second dynamic member having third and fourth portions oriented along the third longitudinal axis, wherein the third portion is configured to rotationally couple to the threaded second end of the offset member and includes a second intersecting axis that extends through the third portion at an angle to the third longitudinal axis and along the first longitudinal axis of the threaded second end to intersect the center point, wherein the fourth portion is configured to slideably receive the second portion, and wherein the first and second dynamic members are configured to maintain the intersection of the first and second intersecting axes with the center point as the center point moves along a curved three dimensional surface during movement of the first dynamic member relative to the second dynamic member.

6. The dynamic stabilization system of claim 5 wherein the shaped first portion is substantially spherical.

7. The dynamic stabilization system of claim 5 wherein a position of the third portion is adjustable along the threaded second portion.

8. The dynamic stabilization system of claim 5 wherein the first portion is configured to rotationally couple to the second polyaxial head by means of another offset member having a second rod connecting a shaped third portion to a threaded fourth portion, wherein the shaped third portion is coupled to the second polyaxial head and the threaded fourth portion is coupled to the first portion.

9. The dynamic stabilization system of claim 8 wherein a position of the first portion is adjustable along the threaded fourth portion.

10. The dynamic stabilization system of claim 5 wherein the rod is curved.

11. The dynamic stabilization system of claim 5 wherein the first longitudinal axis of the threaded second portion is substantially perpendicular to the second longitudinal axis of the rod.

12. A dynamic stabilization system having an offset member for multiple dynamic devices comprising:
an offset member having a rod with a first end coupled to a first polyaxial head, a second end coupled to a second polyaxial head, and first and second threaded extensions extending substantially perpendicularly to a longitudinal axis of the rod between the first and second ends;
a first dynamic device having a first member rotatably coupled to the first threaded extension and slideably engaged to a second member of the first dynamic device that is coupled to a third polyaxial head, wherein movement of the first member relative to the second member and the offset member defines movement of a first center point along a first curved three dimensional surface; and
a second dynamic device having a third member rotatably coupled to the second threaded extension and slideably engaged to a fourth member of the second dynamic device that is coupled to a fourth polyaxial head, wherein movement of the third member relative to the fourth member and the offset member defines movement of a second center point along a second curved three dimensional surface.

13. The dynamic stabilization system of claim 12 wherein the first and second threaded extensions are positioned on a side of the rod opposite the first and second polyaxial heads.

14. The dynamic stabilization system of claim 12 wherein the rod is curved.

15. The dynamic stabilization system of claim 12 wherein the first and second center points are identical.

16. The dynamic stabilization system of claim 12 wherein the first and second curved three dimensional surfaces are identical.

17. The dynamic stabilization system of claim 12 wherein the second and fourth members are coupled to the third and fourth polyaxial heads, respectively, by means of a second offset member.

18. The dynamic stabilization system of claim 17 wherein the second offset member includes a second rod with a third end coupled to the third polyaxial head, a fourth end coupled to the fourth polyaxial head, and third and fourth threaded extensions coupled to the second and fourth members, respectively.

19. The dynamic stabilization system of claim 12 wherein the second member is coupled to the third polyaxial head by means of a second offset member.

20. The dynamic stabilization system of claim 19 wherein the second offset member includes a second rod connecting a shaped first portion to a threaded second portion, wherein a longitudinal axis of the threaded second portion is angled relative to a longitudinal axis of the second rod, and wherein the shaped first portion is coupled to the third polyaxial head and the threaded second portion is coupled to the second member.
